(19) United States
(12) Patent Application Publication
(10) Pub. No.: US 2020/0057875 A1
(43) Pub. Date: Feb. 20, 2020

(54) On-Chip Random ID Generation

(71) Applicant: University of Newcastle Upon Tyne, Tyne and Wear (GB)

(72) Inventors: Timothy Constandinou, London (GB); Sara Ghoreishizadeh, London (GB)

(73) Assignee: University of Newcastle Upon Tyne, Tyne and Wear (GB)

(21) Appl. No.: 16/487,059

(22) PCT Filed: Feb. 15, 2018
(86) PCT No.: PCT/EP2018/053776

§ 371 Date: Aug. 19, 2019

(30) Foreign Application Priority Data
Feb. 17, 2017 (GB) 1702623.8

(51) Int. Cl. G06K 7/10; A61B 5/00; A61B 5/07

(52) U.S. Cl. CPC G06K 7/10039; A61B 5/0031; A61B 2562/08; A61B 5/07; A61B 5/0024

(57) ABSTRACT

A method by which a hub unit can discover IDs from a plurality of satellite units across a shared communication link that connects each of the satellite units to the hub unit, each satellite unit having a satellite unit ID, the method comprising the hub unit broadcasting a message over the shared communication link, the message including an ID segment with a string of bits shorter than a bit length of the satellite unit IDs and an ID segment location; and each satellite unit receiving the broadcast message and comparing the ID segment with an ID portion of its own satellite unit ID starting at the ID segment location, if the ID portion for a satellite unit matches the ID segment, the satellite unit transmitting its complete satellite unit ID to the hub unit over the shared communication link.

### FIELD OF THE INVENTION

The present invention relates to on-chip random ID generation for complementary metal-oxide-semiconductor (CMOS) integrated circuits (ICs), multi-device systems employing random on-chip IDs and methods for discovering IDs from multiple devices in a system. The various aspects of the invention have particular (although not exclusive) application for medical implantable systems.

### BACKGROUND

In systems in which a central unit is in data communication with multiple satellite units it is desirable that each satellite unit has a unique ID by which it can be addressed by the central unit. Particularly in applications, such as implantable medical systems, where there is a desire to keep the number of wires to a minimum, the use of unique IDs for the satellite units facilitates communication over a shared communication channel, connected in the same configuration.

Most existing solutions for generating unique IDs in CMOS can be classified in two main categories:
(i) Non-volatile-memory-based such as EEPROM, Flash, and/or one-time-programmable (OTP) elements, e.g. fuses. Here, the ID is written (electrically) to the CMOS IC after fabrication through a programming interface. For example, a set of fuses may be “burnt” to write a pre-determined ID on a specific CMOS IC. The disadvantage of using this method for medical implants is that each IC needs to be individually programmed after fabrication, but before assembly thus increasing effort and ultimately cost.
(ii) Utilising device-mismatch, noise or other CMOS based process-dependant parameters to generate a random ID; an ID generated through this method is also called physically uncloanable functions (PUF). Examples are SRAM-based ID generation methods where the start-up state of a single cell within the memory block is determined by the mismatch between the threshold voltage of transistors. However, the resulting ID may change with temperature, noise and supply voltage of the IC. This is not acceptable for ICs directly involved in medical implants because noise level and supply voltage of these devices are not fully-controllable.

There is a third category that is in effect a combination of the two categories above; during first power up, random mismatch or process dependant parameters are used to generate a random ID that is then burnt into non-volatile memory.

A challenge is thus to generate a unique ID for each CMOS IC such that it doesn't change with time, supply voltage, and noise level and requires minimal (or ideally no) CMOS post-processing prior to device assembly and deployment (i.e. implantation).

More recently, Fang Tang et al¹ have proposed using the antenna-effect to generate random IDs on CMOS chips. Antenna-effect, more properly known as process plasma-induced damage, is a well-known failure mechanism in CMOS fabrication. The antenna-effect is normally highly undesirable causing permanent damage to transistor gate-oxides during CMOS fabrication. During dry (e.g. plasma) etching the electrical field across the gate oxide increases when electrical charge is accumulated during etching of the extended polysilicon gate and/or connected metal conducting layers. When the electric field reaches a threshold value, Eₜₕ, the gate oxide breaks down. This phenomena reduces the energy gap between the gate and source/drain of the transistor, enabling more charge to tunnel through the gate which in turn reduces the impedance of the gate. In extreme cases, this phenomena causes a full destruction of the gate, leaving a relatively low resistance between the gate and substrate. ¹ Fang Tang, Denis G. Chen, Bo Wang, Amine Bermak, Abbes Amira, and Saqib Mohamad, “CMOS On-Chip Stable True-Random ID Generation Using Antenna Effect”, IEEE ELECTRON DEVICE LETTERS, VOL. 35, NO. 1, JANUARY 2014.

Fang tang et al have proposed a CMOS on-chip ID generation scheme making use of the antenna-effect during fabrication to intentionally break down one gate in a transistor pair, referred to as an “ID pair” (see FIG. 4). As seen in the transistor level schematic of FIG. 4, the ID pair includes two sub-cells in which NMOS transistor gates (M1, M5) are connected together to ground. Looking at the right-hand sub-cell, PMOS transistor (M2) can sink a small leakage current and a digital inverter (M3, M4) is used to amplify and buffer the digital ID number. In the case where NMOS gate M1 is broken down, the diffusion region of the NMOS transistor is shorted to the ground, thus the digital output OUT1 is ‘1’. If the NMOS transistor gate M1 is not broken down ( ), the PMOS transistor will pull up the internal node and as a result, OUT1 is ‘0’. This output (‘1’ or ‘0’) provides one bit of the ID. Fang Tang et al propose that 64 of ID pairs are used to provide the on-chip ID.

### SUMMARY OF THE INVENTION

In a first aspect, the invention proposes an approach to CMOS random on-chip ID generation, in which the antenna-effect is used during chip fabrication to intentionally damage a random one of two sub-cells in each of a plurality of bit ID-generation blocks on the CMOS chip, such that in each bit ID-generation block there is a voltage differential between outputs of the two sub-cells. The polarity of this voltage differential determines whether the bit value for the bit ID-generation block is a ‘1’ or a ‘0’. The bit values from all of the plurality of bit ID-generation blocks define the on-chip ID for the CMOS chip.

By adopting this approach, similarly to Fang Tang et al, there is no need for any pre-implantation post-processing, programming, or calibration of the system to establish unique IDs for each identical device (where for example, multiple identical implants are connected in parallel). Generated IDs are permanent and not dependant on temperature, supply voltage, and/or noise in the system. Unlike the proposal by Fang Tang et al, however, the proposal here does not assume a full-breakdown of an NMOS gate during the fabrication process; by using the voltage differential between outputs of the two sub-cells, the ID bit can be created when a gate in one of the sub-cells is damaged but not completely broken down. This is significant because in mature CMOS technologies antenna-effect tends to cause only a partial-gate-breakdown.

When we refer herein to gate breakdown in one sub-cell of a bit ID-generation block (and possibly by implication no breakdown of the corresponding gate in the other sub-cell) it should be understood to also include the case where there is a degree of breakdown of the gates in both sub-cells but that the breakdown of the gate in one sub-cell is greater than that in the other, so as to cause the differential voltage between the outputs.

More specifically, there is proposed a CMOS circuit for use in generating an on-chip ID, the circuit comprising:
a bit ID-generation block including a pair of sub-cells, each sub-cell having an output node and an input;
wherein the sub-cells are configured such that following antenna-effect damage to one of the sub-cells during fabrication, application of a supply voltage to the inputs will result in a voltage differential between the output nodes of the pair of sub-cells;
the bit ID-generation block further comprising a comparator connected to the output nodes of the pair of sub-cells and configured to output a bit ID value based on the differential voltage between the output nodes of the sub-cells.

In some embodiments, the comparator will be configured so that its output is either equal to the supply voltage (or some other non-zero value), interpreted as a bit value of ‘1’, or is zero volts, interpreted as a bit value of ‘0’.

The CMOS circuit can include a plurality (M) of bit ID-generation blocks as defined above in order to generate an on-chip ID having M bits. Whilst for some applications, as few as two bit ID-generation blocks may be sufficient, typically there will be 10 or more bit ID-generation blocks, or 20 or more bit ID-generation blocks, or 30 or more bit ID-generation blocks.

In some embodiments, the damage to the sub-cell is damage to a gate within the sub-cell, e.g. an NMOS gate.

In some embodiments, each sub-cell of the pair of sub-cells in the bit ID-generation block comprises:
an NMOS transistor and a PMOS transistor, with the source and drain of the NMOS transistor being connected together and to the drain of the PMOS transistor;
the gate of the NMOS transistor being connected to the gate of the NMOS transistor of the other sub-cell of the pair and connected to ground;
the gate and drain of the PMOS transistor being connected to the sub-cell input; and
the sub-cell output node being on the connection between the PMOS transistor drain and the NMOS transistor source.

The sub-cells of each pair are identical to one another. Where the circuit includes a plurality of bit ID-generation blocks (as would normally be the case), all of the blocks are preferably identical to one another.

The comparator may be a fully-differential cross-coupled comparator.

In some embodiments, for each sub-cell there is a switch between the output node and the comparator by which the comparator can be disconnected from the sub-cells, for example during start-up. Beneficially, this can reduce energy consumption during start-up, as the voltage builds at the output nodes of the sub-cells.

Also provided in the context of this first aspect is an implantable system comprising a plurality of implantable devices, each device including a CMOS chip having a CMOS circuit according the first aspect above for generating an on-chip ID for the implantable device. The system may also include a control unit that communicates over a shared communication link (wired or wireless) with the plurality of implantable devices, addressing each device using its associated ID. The control unit may also be implantable.

In a second aspect, the invention provides a method by which a hub unit can discover IDs from a plurality of satellite units across a shared communication link that connects each of the satellite units to the hub unit, each satellite unit having a satellite unit ID, the method comprising:
the hub unit broadcasting a message over the shared communication link, the message including an ID segment with a string of bits shorter than a bit length of the unique IDs and an ID segment location (i.e. the location of the start point on the segment within the complete string of bits in the ID); and
each satellite unit receiving the broadcast message and comparing the ID segment with an ID portion of its own satellite unit ID starting at the ID segment location, if the ID portion for a satellite unit matches the ID segment, the satellite unit transmitting its complete satellite unit ID to the hub unit over the shared communication link.

Typically, all of the satellite unit IDs will have the same bit length (i.e. a known number of bits).

The hub unit can receive and store the satellite unit IDs sent from the satellite units over the shared communication link. The hub unit keeps sending broadcast messages, each time changing the ID segment and/or the ID segment location, until all of the satellite unit IDs have been discovered.

The broadcast message may also include the length (i.e. number of bits) of the ID segment.

Even where all of the satellite units have unique IDs, it is possible that two (or more) satellite units have IDs that include the same ID segment at the same location. In this case, both satellite units will return their full IDs over the shared communication link, causing a collision. This can be detected by the hub unit.

In some embodiments, the hub unit detects the bit number in the returned IDs at which the collision has occurred. On its next iteration of sending the broadcast message, the hub unit changes the ID segment location to the collision location. This ensures a different segment of the IDs is checked in the next iteration, in the hope of avoiding a further collision.

The satellite units may be implantable devices, for example components of a neural prosthesis. The hub unit may also be implantable.

By adopting this approach, satellite unit IDs can be discovered (or “readout”) after installation of a system (e.g. after implantation in the case where the satellite units are implantable devices) and without requiring any additional readout devices. A further benefit is that no preliminary calibration is required. This contrasts with, for example, systems using one-time programmable fuses to create IDs, in which the chips need to be first connected to an external device to be “programmed” individually and then connected/configured within the implantable system. Thus, the approach proposed by the present invention provides a significant advantage in reducing the time, cost and complexity of the assembly/manufacturing process.

The shared communication link may be wired or wireless.

In some embodiments, each satellite unit comprises a CMOS chip having a CMOS circuit according the first aspect above for generating the satellite unit ID.

Once all of the satellite unit IDs have been discovered, subsequent communication from the hub unit to each satellite unit can be accomplished using the respective satellite unit ID to address messages. The satellite units ignore any messages that are not addressed to them.

Instead of using the full satellite unit ID when addressing messages, shorter, secondary IDs can be used instead. For example, the hub unit can assign a unique secondary ID to each satellite unit. Alternatively, in the case where it can be established that a specific length ID portion of each satellite unit ID at a specific location in the IDs is unique to each satellite unit, then this ID portion can be used as the secondary ID for each satellite unit.

For facilitate data transmission to the hub unit from the satellite units over the shared communication link, without collision of messages, each satellite unit can be allocated a unique time-frame or a unique frequency for transmission over the shared link. The unique time-frame or frequency may be derived from the satellite unit ID for each satellite unit or from a secondary ID assigned to each satellite unit.

In a third aspect, the invention provides a method for transmitting messages from each of a plurality of satellite units to a hub unit over a shared communication link, each satellite unit having a unique ID and being assigned a unique time-frame in which to transmit messages to the hub unit over the shared communication link or a unique frequency for message transmission over the shared communication link, the unique time-frame or frequency being derived from the unique ID.

This approach can be used to avoid collision of messages without employing complex message transport protocols and, by using the unique ID of each satellite unit to determine the time-frame (or frequency) it is allocated, the set-up is simplified.

### BRIEF DESCRIPTION OF THE DRAWINGS

Embodiments of the invention will now be described by way of example with reference to the accompanying drawings in which:

FIG. 1 schematically illustrates an implantable neural prosthesis system that employs ID-generation blocks in accordance with an embodiment of the present invention;

FIG. 2 shows an exemplary command for ID discovery and corresponding comparison with an ID; and

FIG. 3 shows an exemplary ID interrogation algorithm used for ID discovery.

### DETAILED DESCRIPTION AND FURTHER OPTIONAL FEATURES OF THE INVENTION

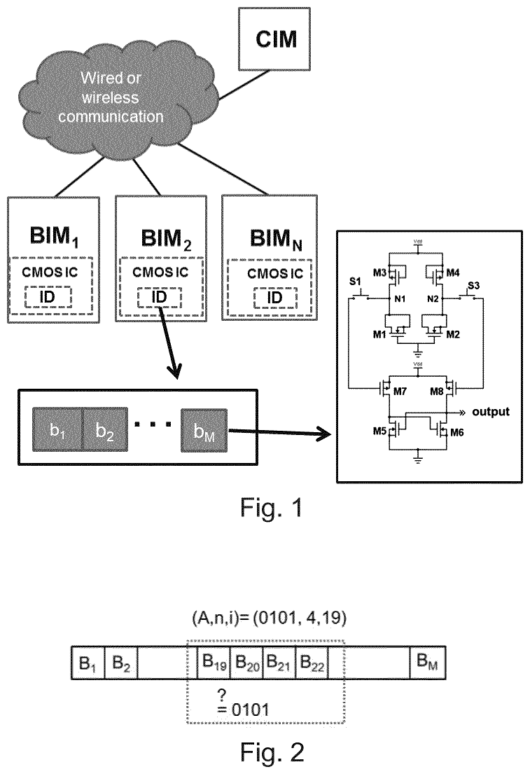

The invention is exemplified with reference to an implantable neural prosthesis, shown in FIG. 1, consisting of N+1 active implanted devices. There is a single Chest Implant (CIM) and N identical Brain Implants (BIM) connected together through a shared wired or wireless communication channel. Each BIM includes an integrated circuit (IC) fabricated in standard CMOS technology.

To enable full-duplex communication between the CIM and multiple BIM's (through the shared communication channel), the BIM's need to be individually addressable by the CIM. Moreover, each BIM should access the channel in a pre-determined time or frequency frame to avoid data collision. The corresponding channel access methods are known as Time-Domain-Multiple-Access (TDMA) and Frequency-Domain-Multiple-Access (FDMA), respectively. To determine the time (or frequency) frame for each of these identical BIM's, it is essential to provide a unique identification code (ID) for each BIM. Once the BIM's are made individually addressable, the channel-access-time (or frequency) window can be selected individually, by the CIM. Alternatively, the ID can be used by the BIM alone to directly determine the time (frequency) frame.

A random, permanent ID is generated in this example by exploiting a well-known failure mechanism in CMOS fabrication called process plasma-induced damage, more commonly known as the antenna-effect, as discussed above.

The proposed circuit to generate a random-bit based on the antenna effect is shown in the box at the right-hand side of FIG. 1 (right). Here, each cell includes two identical sub-cells with NMOS (M1, M2) and PMOS transistors (M3,M4). To ensure the gate-breakdown happens, the peripheral area of the extended-gate-metal of M1 (also connected to M3) is maximised. Moreover, to ensure good randomness, the gates of M1 and M2 are connected together using all metal and polysilicon layers using a perfectly symmetrical design. Therefore, if for example the gate of M1 breaks down, both gates M1 and M2 are discharged and this in turn reduces the electrical field across the gate oxide of M2 to below Eₜₕ so that M2 does not break down. If the layout of both gates is perfectly symmetrical, the probability of breaking one or the other gate is 50%.

All polysilicon and metal layers except the top metal layer are used extend the side-area of M1 (and M3) gate, to maximise the charge pick-up and antenna-effect. The top metal layer is used to connect the gate to ground. The drain and source terminals of the NMOS are connected together and to the drain of a PMOS. The gate and source of the PMOS are tied-up to charge up the drain/source of M1 with a small leakage current.

In the case of a full-breakage in the gate of M1, its source (and drain of M3) is short-circuited to ground and the gate of M2 charges up to VDD. However, in non-“extreme” cases, the damage to the gate only leads to gate oxide degradation resulting in a reduced impedance in the gate of M1 (with respect to M2) and a resistive path through its gate-oxide (i.e. it becomes leaky). This leads to a voltage-division between the output resistance of M3 and the gate-oxide resistance of M1 resulting in a smaller voltage at node N1 than at N2.

Conversely, in the case where M2 is damaged (i.e. M2 becomes leaky) there will be a higher voltage at node N1 than at N2.

In this example, the differential voltage between N1 and N2 is amplified through a comparator (for example, a fully differential cross-coupled comparator as shown in FIG. 1).

During start-up, the voltages of the two nodes charge-up slowly through the leakage current provided by the PMOS transistors. This induces large transient currents in the comparator. In this example, to minimise start-up current (surge), switches S1 and S2 are placed between nodes N1, N2 and the comparator. The switches are open at start-up and can then be closed shortly after (e.g. a few milliseconds after) start-up. Each switch may, for example, be an NMOS transistor that can have “open” and “close” states based on the application of a low (e.g. 0V) or high (e.g. source voltage) voltage applied to its gate.

ID Size Determination

An M-bit ID generation block can be embedded within each BIM to provide a random ID for each BIM. The ID size (M) should be as large as possible to reduce the probability of having two identical IDs generated for two BIMs across a total of M BIMs. However, N should be minimized to save area and dynamic power consumption of the BIM. Here we calculate the probability (P_{eq}) of having the ID of two BIM's equal. The calculations are because each bit is generated completely randomly and independently from all the other bits and the probability of an ID bit being zero or one is exactly equal.

P_{eq}=(M−1) 1/2^{N}+(M−2) 1/2^{N}+(M−3) 1/2^{N}+ . . . +(M−3) 1/2^{N}=M(M−1) 1/2^{(N+1)}

The minimum number of bits in the ID that leads to two equal IDs on two or more different BIM's (among N total BIM's) with a probability of less than α is:

M(M−1) 1/2^{(N+1)}>α→M>(In(M(M−1))−In(α))/In(2)−1

where α can be made arbitrarily small. For example to ensure that the probability of two BIMs (out of 100) end up with identical IDs is less than 10⁻⁸, an ID block with 39-bits is required.

ID Readout Method

To enable the CIM to address the individual BIM's, as well as determining the unique IDs for each BIM, the CIM needs to read and register all IDs in its memory prior to any communication. An ID readout phase is thus required during which all the IDs are scanned and read out, traditionally using an external reader and then saved to non-volatile memory (NVM) of the CIM. This method requires time and extra cost as it consists of an extra readout-step before assembly and implantation of the system.

Here we propose a method for the CIM to read the ID of each BIM through the shared communication channel after assembly and/or implantation. This is based on a polling method that implements a search algorithm with the CIM “guessing” a portion of the ID and requesting the BIMs to respond only if it has successfully guessed a part of its ID.

The CIM includes: (i) non-volatile memory to register IDs of all the optrodes; and (ii) data collision detection.

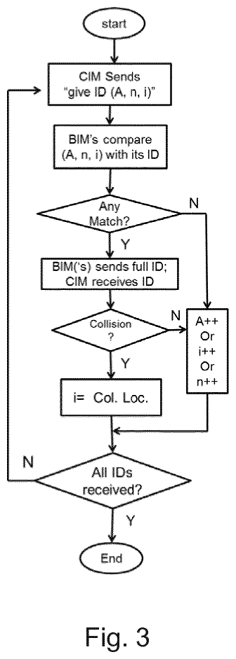

The steps for ID-readout are as follows:
(i) CIM issues a command: “give ID (A,n,i)”to all the BIMs.
(ii) If a BIM successfully matches the received pattern (A,n,i) to its ID, it will respond by sending its full ID.
(iii) The CIM changes the (A,n i) pattern until it receives the IDs from all the BIMs.
(iv) If a collision occurs at bit C, the CIM changes (A,n,i) accordingly.
   where A, n and i are the bit-pattern, the number of bits in the bit-pattern and the location of the bit-pattern in the M-bit ID (See FIG. 2). For example, the following command: “give ID (0101, 4, 12)” asks all the BIMs to search for the 4-bit pattern 0101 at position/bit 12 in its ID, and then reply with its full ID if the pattern matches.

If two (or more) BIMs happen to find (A,n,i), they will both reply with their ID to the CIM. In this case they either have the exact same ID (which is highly unlikely if N is chosen to be relatively large), or they are different in one or more bits, at which a collision happens which can be detected at the CIM.

A corresponding lightweight ID declaration algorithm is embedded within each BIM. The ID declaration on the optrode simply checks for (A,n,i) in its ID and responds only if it finds a match. If it matches it responses with its full ID. The BIM therefore includes a circuit block for comparison and respond.

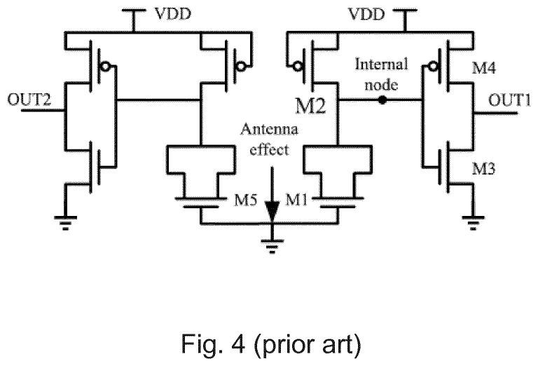

An example of the ID-readout algorithm is shown in FIG. 3.

Dynamic-ID Allocation

As discussed previously, a time (or frequency) frame needs to be allocated for each BIM to share the communication channel. This time frame for a system of 16 BIMs can be implemented using a minimum of 4-bits. This 4-bit frame-identifier is called dynamic ID (DID), and is determined by the CIM and saved in both the BIM and CIM. This number can then be used to address the individual BIM's instead of requiring the long, full M-bit ID. The CIM allocates the DIDs to each BIM after all the IDs are determined.

Adopting this approach, the number of data-bits transmitted can be kept to a minimum. Preferably, all of the time-frames in the TDMA communication are occupied to achieve maximum data-rate.

Secondary Static-ID (SSID) Allocation

An alternative to DID is that CIM extracts an L-bit secondary-static ID (SSID) from the M-bit ID of the BIMs and uses this smaller ID to address the BIMs across the shared communication channel. For example, if all BIMs have and ID in which the first 5-bits are different, a secondary static ID with only 5-bits can be used to address all of them.

The advantage of using the SSID over the dynamic ID is if the system or any of the BIM's loses power and restarts, then the IDs will not be “forgotten”. By saving the SSID location and number of bits in NVM within the CIM, after start-up with a single command all the SSIDs can be established. Whereas for the DID-allocation after each start-up the number of required commands to re-stablish communication is equal to the number of BIMs.

The minimum number of bits needed for the SSID is determined by the number of BIMs. For example, if there are 16 BIMs, only 4 bits are required for the SSID, whereas for a system having 32 BIMs, 5 bits are required. More generally, for a system with N BIMs, the number of bits required for the SSID is log₂(N).

Thus, ff a log₂(N)-bit SSID is found (e.g. using an appropriate algorithm on the IDs for all of the BIMs), the DID allocation phase can be bypassed, and the SSID can be used directly to determine the time-frame (or frequency) for each BIM.

While the invention has been described in conjunction with the exemplary embodiments described above, many equivalent modifications and variations will be apparent to those skilled in the art when given this disclosure. Accordingly, the exemplary embodiments of the invention set forth above are considered illustrative and not limiting. Various changes to the described embodiments may be made without departing from the spirit and scope of the invention.

All references referred to above are hereby incorporated by reference.

1. A method by which a hub unit can discover IDs from a plurality of satellite units across a shared communication link that connects each of the satellite units to the hub unit, each satellite unit having a satellite unit ID, the method comprising:
the hub unit broadcasting a message over the shared communication link, the message including an ID segment with a string of bits shorter than a bit length of the satellite unit IDs and an ID segment location; and
each satellite unit receiving the broadcast message and comparing the ID segment with an ID portion of its own satellite unit ID starting at the ID segment location, if the ID portion for a satellite unit matches the ID segment, the satellite unit transmitting its complete satellite unit ID to the hub unit over the shared communication link.

2. A method according to claim 1, in which all of the satellite unit IDs have the same bit length.

3. A method according to claim 1, wherein the hub unit receives and stores the satellite unit IDs sent from the satellite units over the shared communication link.

4. A method according to claim 1, wherein the hub unit keeps sending broadcast messages, each time changing the ID segment and/or the ID segment location, until all of the satellite unit IDs have been discovered.

5. A method according to claim 1, wherein the broadcast message includes the length of the ID segment.

6. A method according to claim 1, wherein the hub unit detects a collision when two or more satellite units have IDs that include the same ID segment at the same location and both satellite units will return their full IDs over the shared communication link.

7. A method according to claim 6, wherein the hub unit detects a collision location as the bit number in the returned IDs at which the collision has occurred and, on its next iteration of sending the broadcast message, the hub unit changes the ID segment location to the collision location.

8. A CMOS circuit for use in generating an on-chip ID, the circuit comprising:
a bit ID-generation block including a pair of sub-cells, each sub-cell having an output node and an input;
wherein the sub-cells are configured such that following antenna-effect damage to one of the sub-cells during fabrication, application of a supply voltage to the inputs will result in a voltage differential between the output nodes of the pair of sub-cells;
the bit ID-generation block further comprising a comparator connected to the output nodes of the pair of sub-cells and configured to output a bit ID value based on the differential voltage between the output nodes of the sub-cells.

9. A CMOS circuit according to claim 8, wherein the comparator is configured so that its output is either equal to the supply voltage, interpreted as a bit value of ‘1’, or is zero volts, interpreted as a bit value of ‘0’.

10. A CMOS circuit according to claim 8, comprising a plurality (M) of bit ID-generation blocks to generate an on-chip ID having M bits,
each bit ID-generation block including a pair of sub-cells, each sub-cell having an output node and an input;
wherein for each bit ID-generation block the pair of sub-cells are configured such that following antenna-effect damage to one of the sub-cells during fabrication, application of a supply voltage to the inputs will result in a voltage differential between the output nodes of the pair of sub-cells;
each bit ID-generation block further comprising a comparator connected to the output nodes of the pair of sub-cells and configured to output a bit ID value based on the differential voltage between the output nodes of the sub-cells.

11. A CMOS circuit according to claim 8, wherein each sub-cell of the pair of sub-cells in the or each bit ID-generation block comprises:
an NMOS transistor and a PMOS transistor, with the source and drain of the NMOS transistor being connected together and to the drain of the PMOS transistor;
the gate of the NMOS transistor being connected to the gate of the NMOS transistor of the other sub-cell of the pair and connected to ground;
the gate and drain of the PMOS transistor being connected to the sub-cell input; and
the sub-cell output node being on the connection between the PMOS transistor drain and the NMOS transistor source.

12. A CMOS circuit according to claim 11, wherein the damage to the sub-cell is damage to its NMOS gate.

13. A CMOS circuit according to claim 8, wherein for each sub-cell there is a switch between the output node and the comparator by which the comparator can be disconnected from the sub-cells.

14. An implantable system comprising a plurality of implantable devices, each implantable device including a CMOS chip having a CMOS circuit according to claim 8.

15. An implantable system according to claim 14, comprising a control unit and a shared communication link over which the control unit can communicate with the plurality of implantable devices.

16. An implantable system according to claim 15, wherein the control unit is configured to send messages to the plurality of implantable devices over the shared communication link and to address each message to a specific implantable device using the on-chip ID of said specific implantable device.

17. A method according to claim 1, wherein each satellite unit comprises a CMOS chip having a CMOS circuit for generating the satellite unit ID, the CMOS circuit comprising:
a bit ID-generation block including a pair of sub-cells, each sub-cell having an output node and an input;
wherein the sub-cells are configured such that following antenna-effect damage to one of the sub-cells during fabrication, application of a supply voltage to the inputs will result in a voltage differential between the output nodes of the pair of sub-cells;
the bit ID-generation block further comprising a comparator connected to the output nodes of the pair of sub-cells and configured to output a bit ID value based on the differential voltage between the output nodes of the sub-cells.

18. A method according to claim 1, wherein the hub unit assigns a unique secondary ID to each satellite unit, each unique secondary ID having a shorter bit length than the satellite unit ID of the respective satellite unit.

19. A method according to claim 1,
wherein a specific length ID portion of each satellite unit ID at a specific location in the IDs is used as a secondary ID for each satellite unit, and/or
wherein each satellite unit is allocated a unique time-frame or a unique frequency for transmission over the shared link, the unique time-frame or frequency being derived from the satellite unit ID for each satellite unit or from a secondary ID assigned to each satellite unit.

20. (canceled)

21. A method for transmitting messages from each of a plurality of satellite units to a hub unit over a shared communication link, each satellite unit having a unique ID and being assigned a unique time-frame in which to transmit messages to the hub unit over the shared communication link or a unique frequency for message transmission over the shared communication link, the unique time-frame or frequency being derived from the satellite unit's unique ID.
